(19) United States
(12) Patent Application Publication
(10) Pub. No.: US 2021/0321992 A1
(43) Pub. Date: Oct. 21, 2021

(54) DEVICES AND METHODS FOR OBTAINING BIOPSY SAMPLES

(71) Applicant: Covidien LP, Mansfield, MA (US)

(72) Inventors: Nikolai D. Begg, Wellesley, MA (US); Chad A. Pickering, Woburn, MA (US); Dalia P. Leibowitz, Cambridge, MA (US); Timothy J. Wood, Wilmington, MA (US); Andrew P. Bolognese, Waltham, MA (US)

(21) Appl. No.: 16/852,916

(22) Filed: Apr. 20, 2020

(51) Int. Cl. A61B 10/02

(52) U.S. Cl. CPC A61B 10/0233

(57) ABSTRACT

A tissue biopsy device includes an inner drive assembly, an outer tube, and an inner cutting member extending through the outer tube. The inner drive assembly is coupled to both the outer tube and the inner cutting member and is configured to receive a rotational input. In response to receiving the rotational input, the inner drive assembly is configured to alternatingly distally translate the outer tube and the inner cutting member.

### BACKGROUND

### Technical Field

The present disclosure relates generally to surgical devices and methods. More particularly, the present disclosure relates to devices and methods for obtaining, biopsy samples e.g., to diagnose adenomyosis.

### Background of Related Art

Tissue biopsy is a medical procedure used to obtain a tissue sample from an area of the body. The obtained tissue sample may be tested to assist in diagnosing a medical condition or to assess the effectiveness of a particular treatment.

Adenomyosis is a condition in which the inner lining of the uterus, the endometrial tissue, grows into the uterine wall, causing bleeding, cramping, pain, or other complications. Presently, the diagnosis of adenomyosis is difficult because there is no definitive test for diagnosing adenomyosis; complicating matters further, symptoms of adenomyosis are similar to those of other conditions.

### SUMMARY

As used herein, the term “distal” refers to the portion that is described which is farther from a user, while the term “proximal” refers to the portion that is being described which is closer to a user. The terms “substantially” and “approximately,” as utilized herein, account for industry-accepted material, manufacturing, measurement, use, and/or environmental tolerances. Further, any or all of the aspects and features described herein, to the extent consistent, may be used in conjunction with any or all of the other aspects and features described herein.

Provided in accordance with aspects of the present disclosure is a tissue biopsy device including an inner drive assembly, an outer tube, and an inner cutting member extending through the outer tube. The inner drive assembly includes a drive member configured to receive a rotational input and to rotate in response thereto. The drive member defines a groove along at least a portion of a length thereof that has a continuous configuration including alternating helical segments and annular segments. The inner drive assembly further includes first and second drive couplers each at least partially engaged within the groove such that the first and second drive coupler are moved through the groove upon rotation of the drive member. The first and second drive couplers are spaced-apart from one another such that when one of the first or second drive couplers is disposed within one of the helical segments of the groove, the other of the first or second drive couplers is disposed within one of the annular segments of the groove. The first and second drive couplers are driven to translate longitudinally when moving through one of the helical segments and retained in longitudinal position when moving through one of the annular segments such that the first and second drive couplers are alternatingly translated longitudinally. The outer tube is coupled to the second drive coupler such that translation of the second drive coupler translates the outer tube while the inner cutting member is coupled to the first drive coupler such that translation of the first drive coupler translates the inner cutting member.

In an aspect of the present disclosure, at least one of the outer tube or the inner cutting member is coupled to the drive member such that rotation of the drive member drives rotation of the at least one of the outer tube or the inner cutting member.

In another aspect of the present disclosure, the tissue cutting member includes a proximal support and a spiral-shaped distal portion extending distally from the proximal support.

In still another aspect of the present disclosure, the device further includes a handle housing supporting a drive assembly therein. The drive assembly is configured to connect to the inner drive assembly and to provide the rotational input thereto. In such aspects, the drive assembly may include a motor configured to provide the rotational input. Alternatively, a manual actuator coupled to the drive assembly may be provided such that actuation of the manual actuator drives the drive assembly to provide the rotational input.

Another tissue biopsy device provided in accordance with the present disclosure includes a drive extension defining a distal collar, a stepped clutch, an outer tube, and an inner cutting member extending through the outer tube. The stepped clutch includes a housing defining an internal cavity and having a plurality of longitudinally-spaced slots defined on an internal wall of the housing surrounding the cavity. The drive extension extends into the internal cavity such that the distal collar is disposed within the internal cavity. The stepped-clutch further includes a plate disposed within the housing distally of the distal collar and engaged with a first slot of the plurality of longitudinally-spaced slots to retain the plate in position. A biasing member is disposed between the distal collar and the plate. The outer tube is coupled to the plate such that translation of the plate translates the outer tube. The inner cutting member is coupled to the drive extension such that translation of the drive extension translates the inner cutting member. Translation of the drive extension towards the plate loads the biasing member until a sufficient force is achieved that overcomes a retention force of the plate within the first slot such that the biasing member urges the plate to disengage from the first slot, translate distally, and engage with a second slot of the plurality of longitudinally-spaced slots.

In an aspect of the present disclosure, the device further includes a drive member configured to receive a rotational input and to rotate in response thereto. The outer tube and the inner cutting member are both coupled to the drive member such that rotation of the drive member drives rotation of the outer tube and the inner cutting member.

In another aspect of the present disclosure, the drive member is further configured to translate in response to receiving the rotational input. In such aspects, the drive extension is coupled to the drive member such that translation of the drive member drives translation of the drive extension.

In yet another aspect of the present disclosure, the drive member includes a helical groove along at least a portion of a length thereof and a drive coupler is at least partially engaged within the helical groove such that the drive member is translated longitudinally in response to rotation of the drive member.

In still another aspect of the present disclosure, the device further includes a handle housing supporting a drive assembly therein. The drive assembly is configured to provide the rotational input to the drive member. In such aspects, the drive assembly may include a motor configured to provide the rotational input. Alternatively, a manual actuator coupled to the drive assembly may be provided such that actuation of the manual actuator drives the drive assembly to provide the rotational input.

In still yet another aspect of the present disclosure, the tissue cutting member includes a proximal support and a spiral-shaped distal portion extending distally from the proximal support.

Another tissue biopsy device provided in accordance with the present disclosure includes an inner drive assembly, an outer tube, and an inner cutting member extending through the outer tube. The inner drive assembly includes a drive member configured to receive a rotational input and to rotate in response thereto. The drive member defines a helical groove along at least a portion of a length thereof. The drive member includes a protrusion extending distally therefrom that is off-center from a longitudinal axis of the drive member such that the protrusion orbits about the longitudinal axis upon rotation of the drive member about the longitudinal axis. The inner drive assembly further includes a drive coupler at least partially engaged within the helical groove such that the drive coupler is moved through the groove upon rotation of the drive member to thereby translate the driver member longitudinally. An oblong cam lobe is positioned distally adjacent the drive coupler and is pivotable about a pivot pin transversely aligned on the longitudinal axis. First and second pushers are positioned distally adjacent the oblong cam lobe. Upon orbiting of the protrusion to a first position, the protrusion urges the oblong cam lobe to pivot in a first direction such that a first end portion of the oblong cam lobe urges the first pusher to translate distally. Upon orbiting of the protrusion from the first position to a second position, the protrusion urges the oblong cam lobe to pivot in a second, opposite direction such that a second end portion of the oblong cam lobe urges the second pusher to translate distally. The outer tube is coupled to the second pusher such that translation of the second pusher translates the outer tube and the inner cutting member is coupled to the first pusher such that translation of the first pusher translates the inner cutting member.

In an aspect of the present disclosure, at least one of the outer tube or the inner cutting member is coupled to the drive member such that rotation of the drive member drives rotation of the at least one of the outer tube or the inner cutting member.

In another aspect of the present disclosure, the tissue cutting member includes a proximal support and a spiral-shaped distal portion extending distally from the proximal support.

In still another aspect of the present disclosure, the device further includes a handle housing supporting a drive assembly therein. The drive assembly is configured to connect to the inner drive assembly and to provide the rotational input thereto. In such aspects, the drive assembly may include a motor configured to provide the rotational input. Alternatively, a manual actuator coupled to the drive assembly may be provided such that actuation of the manual actuator drives the drive assembly to provide the rotational input.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side view of a powered tissue biopsy device provided in accordance with the present disclosure;

FIG. 2 is a side view of a manual tissue biopsy device provided in accordance with the present disclosure;

FIG. 3 is a side, partial cross-sectional view of an inner drive assembly and end effector assembly configured for use with the device of FIG. 1, the device of FIG. 2, or any other suitable tissue biopsy device;

FIGS. 4A-4C are side, partial cross-sectional views of another inner drive assembly and end effector assembly configured for use with the device of FIG. 1, the device of FIG. 2, or any other suitable tissue biopsy device, progressively illustrating use thereof;

FIG. 5 is a side, partial cross-sectional view of yet another inner drive assembly and end effector assembly configured for use with the device of FIG. 1, the device of FIG. 2, or any other suitable tissue biopsy device; and

FIGS. 6A-6C are longitudinal, cross-sectional views progressively illustrating obtaining an adenomyosis biopsy sample in accordance with the present disclosure.

### DETAILED DESCRIPTION

Aspects and features of the present disclosure are described in detail with reference to the drawings, in which like reference numerals designate identical or corresponding elements in each of the several views. In the following description, well-known functions or constructions are not described in detail to avoid obscuring the present disclosure in unnecessary detail.

The devices, systems, and methods of the present disclosure may be used for obtaining a tissue sample during any open, minimally invasive, natural orifice, or other surgical procedure. That is, although the devices and methods of the present disclosure are described below with reference to a myometrial biopsy procedure to diagnose adenornyosis, the systems and methods of the present disclosure may also be used for other suitable tissue biopsy procedures.

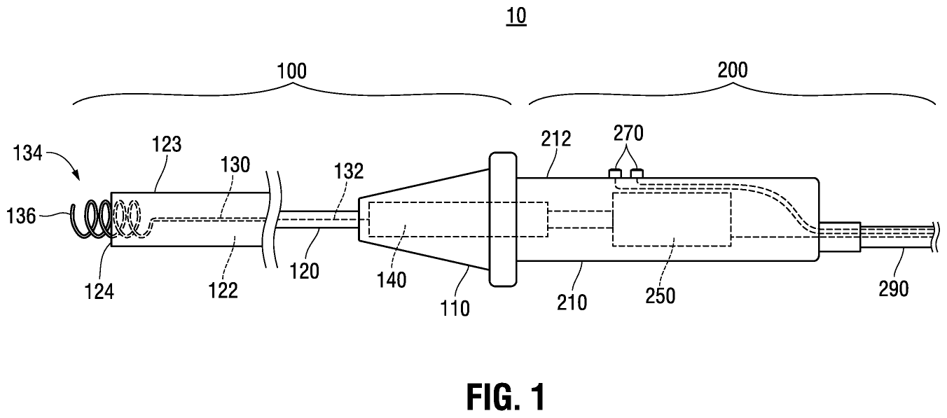

With reference to FIG. 1, a device for obtaining biopsy samples is shown generally identified by reference numeral 10 including an end effector assembly 100 and a handpiece assembly 200. Handpiece assembly 200 generally includes handle housing 210, a motor 250 disposed within handle housing 210, one or more controls 270, e.g., buttons, disposed on handle housing 210 to facilitate activation of device 10, and a cable 290 enabling connection of handpiece assembly 200 to a power source (not shown) or control console including a power source (not shown), although it is also contemplated that handpiece assembly 200 be configured as a battery-powered device, e.g., including a battery and control electronics within handle housing 210.

Handle housing 210 defines a pencil-grip configuration, although other configurations are also contemplated, e.g., pistol-grip configurations, and includes a distal end portion 212 configured to enable operable engagement of end effector assembly 100 with handpiece assembly 200 such that, upon engagement of end effector assembly 100 with handpiece assembly 200, a portion of end effector assembly 100 extends through distal end portion 212 and into handle housing 210 to operably couple with motor 250.

With continued reference to FIG. 1, device 10 may be configured as a single-use instrument that is discarded after use or sent to a manufacturer for reprocessing, a reusable instrument capable of being cleaned and/or sterilized for repeated use by the end-user, or a partially-single-use, partially-reusable instrument. With respect to partially-single-use, partially-reusable configurations, handpiece assembly 200 may be configured as a cleanable/sterilizable, reusable component, while end effector assembly 100 is configured as a single-use, disposable/reprocessable component, or vice versa. In any of the above configurations, end effector assembly 100 may be configured to releasably engage handpiece assembly 200 to facilitate disposal/reprocessing of any single-use components and cleaning and/or sterilization of any reusable components. Further, enabling releasable engagement of end effector assembly 100 with handpiece assembly 200 allows for use of different end effector assemblies 100 with handpiece assembly 200. In other embodiments, end effector assembly 100 is permanently secured to handpiece assembly 200.

End effector assembly 100 includes a proximal hub housing 110 configured to engage handpiece assembly 200, an outer tube 120 extending distally from proximal hub housing 110, and an inner cutting member 130 disposed within and extending through outer tube 120. Outer tube 120 defines a lumen 122 extending therethrough and a distal edge 124 surrounding an open distal end 123 of outer tube 120. Distal edge 124 may define a sharpened configuration about at least a portion of the circumference thereof, one or more angles, one or more chamfers, cutting teeth disposed about at least a portion of the circumference thereof, and/or any other suitable features configured to facilitate cutting of tissue upon rotation and/or translation of distal edge 124 (as a result of rotation of outer tube 120) relative to tissue.

Inner cutting member 130, as noted above, is disposed within and extends through outer tube 120. Inner cutting member 130 includes a proximal support 132 formed as a wire, rod, tube, or in any other suitable manner. Inner cutting member 130 further includes a spiral-shaped distal portion 134 extending distally from proximal support 132. In some configurations, proximal support and spiral-shaped distal portion 134 are integrally formed as a single component, e.g., a continuous piece of wire. Spiral-shaped distal portion 134 may define a sharpened free end 136 or any other suitable configuration of free distal end 136. Spiral-shaped distal portion 134, lead by free distal end 136, is configured to engage and bore into tissue upon rotation of inner cutting member 130 relative thereto, e.g., in a corkscrew-like fashion, in a direction of the spiral of spiral-shaped distal portion 136. On the other hand, spiral-shaped distal portion 134 may be withdrawn from tissue via rotation in the opposite direction, e.g., opposite the direction of the spiral of spiral-shaped distal portion 136.

Inner cutting member 130 may, in an initial position, extend to the distal end of outer tube 120 such that free distal end 136 of spiral-shaped distal portion 134 is disposed adjacent distal edge 124 of outer tube 120; may, in the initial position, extend distally beyond the distal end of outer tube 120 such that at least a portion of spiral-shaped distal portion 134 extends distally from the distal end of outer tube 120; or may, in the initial position, be recessed within outer tube 120 such that free distal end 136 of spiral-shaped distal portion 134 is disposed within lumen 122 of outer tube 120 and proximally-spaced from distal edge 124 of outer tube 120. Inner cutting member 130 and outer tube 120 are configured to translate longitudinally relative to one another (and proximal hub housing 110) and are configured to rotate with one another, although it is also contemplated that inner cutting member and outer tube 120 may rotate oppositely of one another, in sequential or overlapping temporal relation relative to one another, and/or at different speeds relative to one another. The rotation of inner cutting member 130 and outer tube 120 as well as the relative translation between inner cutting member 130 and outer tube 120 may facilitate capturing a biopsy sample therewith, as detailed below.

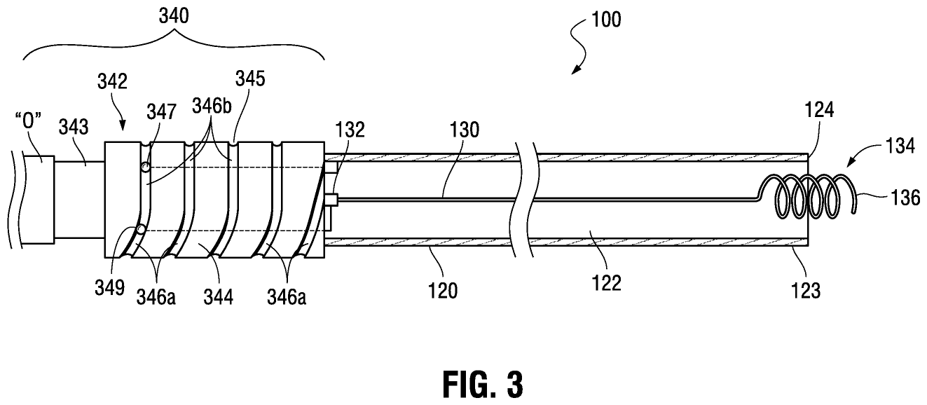

End effector assembly 100 further includes an inner drive assembly 140 at least partially disposed within proximal hub housing 110 and operably coupled to outer tube 120 and inner cutting member 130. Inner drive assembly 140, more specifically, is configured to operably couple motor 250 of handpiece assembly 200 with both outer tube 120 and inner cutting member 130 such that, upon activation of motor 250 (which provides a rotational output, although translational outputs or combination rotational and translational outputs are also contemplated), outer tube 120 and inner cutting member 130 are driven to rotate together with one another, and such that outer tube 120 and inner cutting member 130 are configured to alternatingly translate distally relative to one another and proximal hub housing 110. Various inner drive assemblies 140 suitable for use with device 10 are detailed below with reference to FIGS. 3-5.

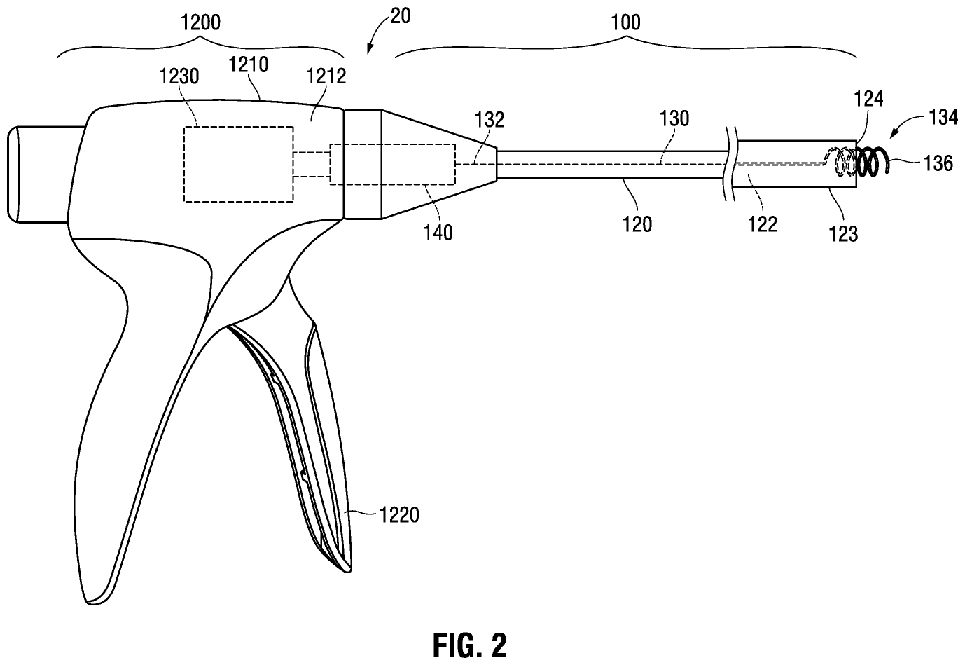

Turning to FIG. 2, another device for obtaining adenomyosis and other biopsy samples is shown generally identified by reference numeral 20 including end effector assembly 100 and a handpiece assembly 1200.

Handpiece assembly 1200 is configured for manual actuation and generally includes a handle housing 1210, a trigger 1220 pivotably coupled to handle housing 1210, and a drive assembly 1230 disposed within handle housing 1210 and operably coupled to trigger 1220. Handle housing 1210 defines a pistol-grip configuration, although other configurations are also contemplated; further, rather than providing a pivoting trigger 1220 to actuate handpiece assembly 1200, handpiece assembly 1200 may include one or more slidable plungers, buttons, etc. A distal end portion 1212 of handle housing 1210 is configured to enable operable engagement of end effector assembly 100 with handpiece assembly 1200 such that, upon engagement of end effector assembly 100 with handpiece assembly 1200, a portion of end effector assembly 100 extends through distal end portion 1212 and into handle housing 1210 to operably couple with drive assembly 1230.

With continued reference to FIG. 2, device 20 may be configured as a single-use instrument that is discarded after use or sent to a manufacturer for reprocessing, a reusable instrument capable of being cleaned and/or sterilized for repeated use by the end-user, or a partially-single-use, partially-reusable instrument. With respect to partially-single-use, partially-reusable configurations, handpiece assembly 1200 may be configured as a cleanable/sterilizable, reusable component, while end effector assembly 100 is configured as a single-use, disposable/reprocessable component, or vice versa. In any of the above configurations, end effector assembly 100 may be configured to releasably engage handpiece assembly 1200 to facilitate disposal/reprocessing of any single-use components and cleaning and/or sterilization of any reusable components. Further, enabling releasable engagement of end effector assembly 100 with handpiece assembly 1200 allows for use of different end effector assemblies 100 with handpiece assembly 1200. In other embodiments, end effector assembly 100 is permanently secured to handpiece assembly 1200.

End effector assembly 100 is detailed above with respect to device 10 (FIG. 1) and, thus, the description thereof is not repeated with respect to device 20 except as necessary to detail the use of end effector assembly 100 with handpiece assembly 1200.

Drive assembly 1230 of handpiece assembly 1200 is configured and operably coupled to trigger 1220 such that actuation of trigger 1220, e.g., pivoting of trigger 1220 relative to handle housing 1210, actuates drive assembly 1230 to provide a rotational output, although in embodiments, translational outputs and/or combination rotational and translational outputs are also contemplated. Inner drive assembly 140 of end effector assembly 100 is configured to operably couple drive assembly 1230 of handpiece assembly 1200 with both outer tube 120 and inner cutting member 130 such that, upon activation of drive assembly 1230, e.g., in response to actuation of trigger 1220, outer tube 120 and inner cutting member 130 are driven to rotate together with one another, and such that outer tube 120 and inner cutting member 130 are configured to alternatingly translate distally relative to one another and proximal hub housing 110. Various inner drive assemblies 140 suitable for use with device 20 are detailed below with reference to FIGS. 3-5.

Turning to FIG. 3, an inner drive assembly configured for use with end effector assembly 100 is shown generally identified by reference numeral 340. Inner drive assembly 340 includes a proximal driver 342 and first and second drive couplers 347, 349, respectively. Proximal driver 342 includes a proximal extension 343 that is coupled to and configured to receive a rotational driving force from a rotational output “O,” e.g., the rotational output of motor 250 (FIG. 1), the rotational output of drive assembly 1230 (FIG. 2), or any other suitable rotational output “O.” The rotational output “O” drives rotation of proximal extension 343 of proximal driver 342. Proximal driver 342 further includes a distal sleeve 344 integrally formed with, engaged with, or otherwise coupled to proximal extension 343 in fixed orientation and position relative thereto, e.g., such that rotation/translation of proximal extension 343 rotates/translates distal sleeve 344.

Distal sleeve 344 defines a groove 345 extending along at least a portion of a length thereof. Groove 345 may be defined on an outwardly-facing surface of distal sleeve 344, an inwardly-facing surface of distal sleeve 344, or may be configured as a slot extending completely through distal sleeve 344. Groove 345 defines a continuous configuration and includes alternating helical segments 346a and annular segments 346b. The helical segments 346a may define constant or variable pitches that are similar to or different from one another. Further, groove 345 may be configured to enable uni-directional motion, e.g., wherein groove 345 includes just a forward portion, or may be configured for bi-directional or reciprocal motion, e.g., wherein groove 345 defines forward and reverse portions. With respect to bi-directional configurations, the ends of the forward and reverse portions may be blended to enable transition from one translational direction to the other.

First and second drive couplers 347, 349 may be configured as drive pins, drive cams, or other suitable drive structures and are at least partially disposed within groove 345 in spaced-apart relation relative to one another. More specifically, first and second drive couplers 347, 349 are spaced-apart from one another such that when first drive coupler 347 is disposed within a helical segment 346a of groove 345, second drive coupler 349 is disposed within an annular segments 346b of groove 345 and such that when first drive coupler 347 is disposed within an annular segment 346b of groove 345, second drive coupler 349 is disposed within a helical segments 346a of groove 345. First and second drive couplers 347, 349 are rotationally fixed relative to proximal hub housing 110 (FIG. 1) of end effector assembly 100 but permitted to translate relative thereto such that, in response to rotation of distal sleeve 344, first and second drive couplers 347, 349 are moved along groove 345.

As first drive coupler 347 is moved along groove 345 in response to rotation of distal sleeve 344, first drive coupler 347 is translated distally while disposed within any of the helical segments 346a of groove 345 and is maintained in position (longitudinally) while disposed within any of the annular segments 346b of groove 345. Likewise, as second drive coupler 349 is moved along groove 345 in response to rotation of distal sleeve 344, second drive coupler 349 is translated distally while disposed within any of the helical segments 346a of groove 345 and is maintained in position (longitudinally) while disposed within any of the annular segments 346b of groove 345. With first and second drive couplers 347, 349 not occupying the same type of segment 346a, 346b, the result is that second drive coupler 349 is maintained in longitudinal position while first drive coupler 347 is translated distally and that first drive coupler 347 is maintained in longitudinal position while second drive coupler 349 is translated distally. In other words, first and second drive couplers 347, 349 are translated distally in alternating fashion.

Continuing with reference to FIG. 3, a proximal end portion of proximal support 132 of inner cutting member 130 is coupled with proximal driver 342 in fixed rotational orientation, e.g., such that rotational driving of proximal extension 343 (via the a rotational output “O,” for example) drives rotation of inner cutting member 130. Inner cutting member 130, however, is slidably coupled with proximal driver 342 to enable translation of inner cutting member 130 relative thereto. This or any other slidable, fixed rotational coupling of the present disclosure may be provided via a pin-longitudinal slot engagement or other suitable direct or indirect engagement. The proximal end portion of proximal support 132 of inner cutting member 130 is, on the other hand, longitudinally fixed relative to first drive coupler 347 but is rotatable relative thereto. In this manner, translation of first drive coupler 347 translates inner cutting member 130 while permitting rotation of inner cutting member 130 relative thereto. This or any other rotational, fixed translational coupling of the present disclosure may be provided via a pin-annular slot engagement or other suitable direct or indirect engagement.

A proximal end portion of outer tube 120, similar to proximal support 132 of inner cutting member 130, is coupled with proximal driver 342 in fixed rotational orientation, e.g., such that rotational driving of proximal extension 343 (via the a rotational output “O,” for example) drives rotation of outer tube 120. Outer tube 120, however, is slidably coupled with proximal driver 342 to enable translation of outer tube 120 relative thereto. The proximal end portion of outer tube 120 is, on the other hand, longitudinally fixed relative to second drive coupler 349 but is rotatable relative thereto. In this manner, translation of second drive coupler 349 translates outer tube 120 while permitting rotation of outer tube 120 relative thereto.

As a result of the above-detailed rotationally-fixed couplings of proximal extension 343 with distal sleeve 344, outer tube 120, and inner cutting member 130, rotational driving of proximal extension 343 (via the a rotational output “O,” for example) drives similar rotation of distal sleeve 344, outer tube 120, and inner cutting member 130. Further, as a result of the fixed translational couplings of first and second driver couplers 347, 349 with inner cutting member 130 and outer tube 120, respectively, the rotation of distal sleeve 344 alternatingly translates inner cutting member and outer tube 120, as detailed above, relative to one another and relative to proximal hub housing 110 (FIG. 1). Thus, in use, during a first portion of activation, inner cutting member 130 is rotated and advanced distally to engage tissue while outer tube 120 is rotated but maintained in longitudinal position and, during a second portion of activation, outer tube 120 is rotated and advanced distally to cut tissue surrounding inner cutting member 130 and receive a sample of tissue within lumen 122 of outer tube 120 while inner cutting member 130 is rotated but maintained in longitudinal position to facilitate the cutting of the sample of tissue.

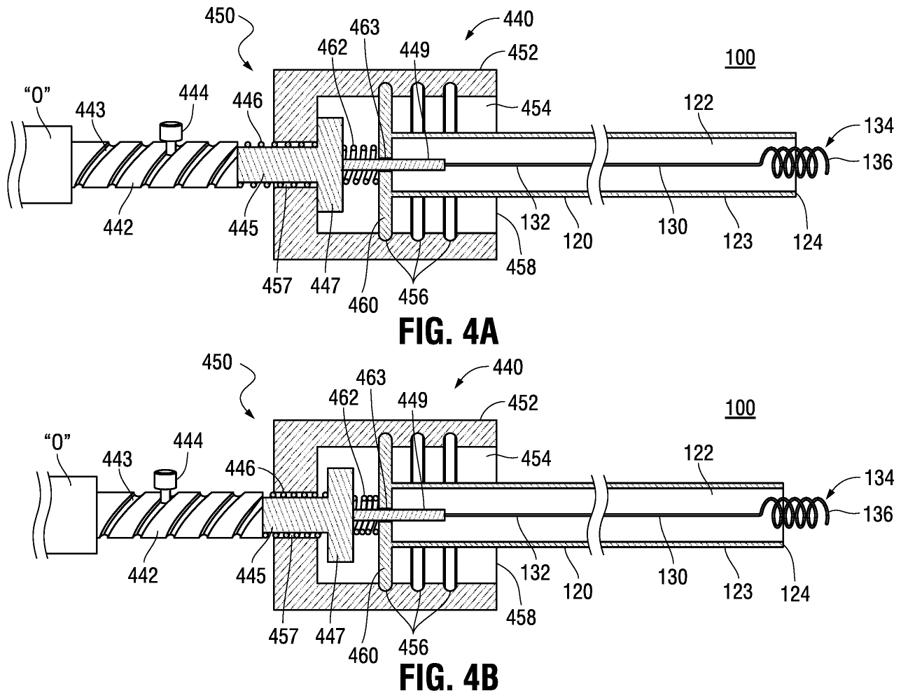

Referring to FIGS. 4A-4C, another inner drive assembly configured for use with end effector assembly 100 is shown generally identified by reference numeral 440. Inner drive assembly 440 includes a proximal driver 442, a drive coupler 444, and a stepped clutch mechanism 450. Proximal driver 442 is coupled to and configured to receive a rotational driving force from a rotational output “O,” e.g., the rotational output of motor 250 (FIG. 1), the rotational output of drive assembly 1230 (FIG. 2), or any other suitable rotational output “O,” although translational outputs and/or combination rotational and translational outputs are also contemplated. The rotational output “O” drives rotation of proximal driver 442. Proximal driver 442 defines a helical groove 443 extending along at least a portion of a length thereof. Helical groove 443 may define a constant or variable pitch, and may be a single helix or a double helix for uni-directional or reciprocal motion, respectively. With respect to a double helix, the ends may be blended to enable transition from one translational direction to the other. Drive coupler 444 is substantially fixed and is at least partially disposed within helical groove 443. In this manner, rotational driving of proximal driver 442 also results in translation of proximal driver 442 as drive coupler 444 travels through helical groove 443.

Proximal driver 442 is slidably disposed, in fixed rotational orientation, about a distal extension 445 which extends distally from proximal driver 442. A biasing member 446, e.g., a coil spring, is disposed about distal extension 445 between proximal driver 442 and a distal collar 447 of distal extension 445 to bias proximal driver 442 proximally relative to distal extension 445. Distal extension 445 includes a distal rod 449 extending therefrom that is fixedly engaged, directly or indirectly, with a proximal end portion of proximal support 132 of inner cutting member 130 such that rotation and translation of distal extension 445 are imparted to inner cutting member 130.

Stepped clutch mechanism 450 includes a housing 452 defining an internal cavity 454 and a plurality of longitudinally-spaced slots 456 defined within an internal surface of housing 452 that surrounds cavity 454. Housing 452 defines a proximal opening 457 and an open distal end 458. Distal extension 445 extends through proximal opening 457 and into cavity 454 of housing 452 such that distal collar 447 is disposed within cavity 454. Stepped clutch mechanism 450 further includes a transversely-oriented plate 460 disposed within cavity 454. Plate 460 is initially engaged with a proximal-most slot 456 of the plurality of longitudinally-spaced slots 456 such that translation of plate 460 relative to housing 452 is inhibited. A biasing member 462, e.g., a coil spring, is disposed within cavity 454 between distal collar 447 and plate 460 to bias plate 460 distally relative to distal collar 447.

A proximal end portion of outer tube 120 is engaged with plate 460. Further, plate 460 defines a central opening 463 permitting passage of distal rod 449 therethrough to, as noted above, allow engagement of distal rod 449 with inner cutting member 130. Central opening 463 and distal rod 449 may be keyed such that rotation of distal rod 449 likewise rotates plate 460 and, thus, such that rotation of inner cutting member 130 likewise rotates outer tube 120.

Continuing with reference to FIGS. 4A-4C, and first to FIG. 4A, prior to activation, proximal driver 442, distal extension 445, distal rod 449, inner cutting member 130, plate 460, and outer tube 120 are in proximal-most positions. With additional reference to FIG. 4B, upon activation, e.g., upon proximal driver 442 receiving a rotational driving force from rotational output “O,” proximal driver 442 is driven to rotate, thereby rotating distal extension 445, distal rod 449, and inner cutting member 130 and, in devices where plate 460 and distal rod 449 are keyed, also rotating plate 460 and outer tube 120.

In addition to driving rotation, the rotation of proximal driver 442 urges drive coupler 444 through groove 443 such that proximal driver 442 is urged to translate distally. More specifically, proximal driver 442 is slid distally about distal extension 445 to compress biasing member 446 against distal collar 447 and, upon sufficient force being applied, translate distal collar 447 distally through cavity 454. The distal translation of distal collar 447 serves to translate distal rod 449 and, thus, inner cutting member 130 distally. The rotation and translation of inner cutting member 130 facilitates the boring and engagement of inner cutting member 130 within tissue. The translation of inner cutting member 130 may be continuous at a substantially constant speed (except for the beginning acceleration and ending deceleration thereof), although stepped translation of inner cutting member 130 is also contemplated, similarly as detailed below with respect to outer tube 120.

Referring also to FIG. 4C, as distal collar 447 is initially translated distally, biasing member 462 is compressed between distal collar 447 and plate 460 such that plate 460 is retained in position, e.g., engaged within the proximal-most slot 456 of the plurality of longitudinally-spaced slots 456. Upon further distal translation of distal collar 447, biasing member 462 is compressed further such that a sufficient potential energy is built up to overcome the retention force of the engagement of plate 460 within the proximal-most slot 456. When this potential energy is achieved, biasing member 462 urges plate 460 to dislodge from the proximal-most slot 456 and slide distally, e.g., into engagement with a next slot 456. As plate 460 is moved distally from one slot 456 to the next slot 456, outer tube 120 is likewise translated distally about and relative to inner cutting member 130. The above is repeated to periodically advance outer tube 120 distally, incrementally advancing plate 460 into engagement with successive slots 456. That is, while inner cutting member 130 is continuously and consistently translated distally, outer tube 120 moves in a stepped manner: periodically translating distally while remaining stationary between the periodic translations. Alternatively, as noted above, inner cutting member 130 may likewise translate in a stepped manner, to alternatingly translate with outer tube 120, to translate in synchronization therewith, or to translate in partially-overlapping temporal relation therewith.

The translation of outer tube 120 (and, in some cases, the rotation thereof) cuts tissue surrounding inner cutting member 130 and captures a sample of tissue within lumen 122 of outer tube 120.

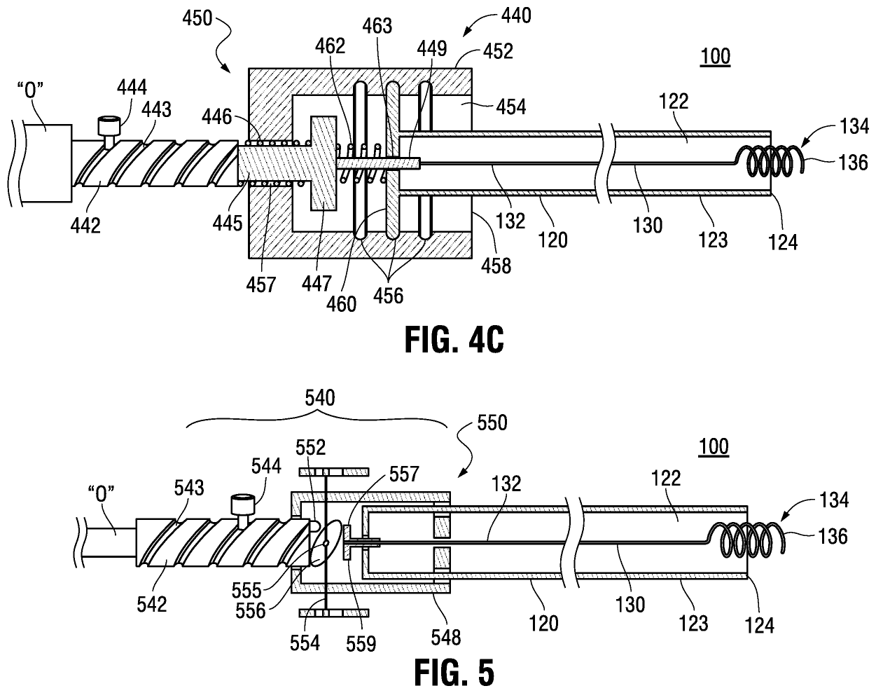

Referring to FIG. 5, still another inner drive assembly configured for use with end effector assembly 100 is shown generally identified by reference numeral 540. Inner drive assembly 540 includes a proximal driver 542, a drive coupler 544, and a cam mechanism 550. Proximal driver 542 is coupled to and configured to receive a rotational driving force from a rotational output “O,” e.g., the rotational output of motor 250 (FIG. 1), the rotational output of drive assembly 1230 (FIG. 2), or any other suitable rotational output “O,” although translational outputs and/or combination rotational and translational outputs are also contemplated. The rotational output “O” drives rotation of proximal driver 542. Proximal driver 542 defines a helical groove 543 extending along at least a portion of a length thereof. Helical groove 543 may define a constant or variable pitch, and may be a single helix or a double helix for uni-directional or reciprocal motion, respectively. With respect to a double helix, the ends may be blended to enable transition from one translational direction to the other. Drive coupler 544 is substantially fixed and is at least partially disposed within helical groove 543. In this manner, rotational driving of proximal driver 542 also results in translation of proximal driver 542 as drive coupler 544 travels through helical groove 543. Proximal driver 542 extend distally into a housing 548 and is slidable and rotatable relative thereto. Housing 548 is fixed relative to proximal driver 542 such that housing 548 translates and rotates therewith. Further, housing 548 is rotationally fixed about a proximal end portion of outer tube 120 and a proximal end portion of proximal support 132 of inner cutting member 130 such that rotation of housing 548, e.g., in response to rotation of proximal driver 542, rotates both outer tube 120 and inner cutting member 130. However, housing 548 is slidable about and relative to outer tube 120 and inner cutting member 130 such that translation of housing 548 is not imparted to outer tube 120 or inner cutting member 130.

Cam mechanism 550 is disposed within housing 548 and includes a protrusion 552, a clevis 554, and an oblong cam lobe 556. Protrusion 552 extends from and is fixed relative to proximal driver 542 at a position offset from a longitudinal axis of proximal driver 542 such that, upon rotation of proximal driver 542, protrusion 552 orbits about the longitudinal axis of proximal driver 542. Clevis 554 supports a pivot pin 555 aligned transversely on the longitudinal axis of proximal drier 542. Oblong cam lobe 556 is rotatably mounted on pivot pin 555. Clevis 554, pivot pin 555, and oblong cam lobe 556 are rotationally isolated from proximal driver 542, housing 548, outer tube 120, and inner cutting member 130 such that neither clevis 554, pivot pin 555, nor oblong cam lobe 556 is rotated in response to rotation of any of proximal driver 542, housing 548, outer tube 120, and inner cutting member 130. However, clevis 554, pivot pin 555, and oblong cam lobe 556 are translationally coupled with proximal driver 542 such that translation of proximal driver 542 affects similar translation of clevis 554, pivot pin 555, and oblong cam lobe 556.

First and second pushers 557, 559 of cam mechanism 550 are positioned on either side of the longitudinal axis of proximal driver 542 adjacent opposing end portions of oblong cam lobe 556. First and second pushers 557, 559 are rotatably coupled with the proximal end portion of outer tube 120 and the proximal end portion of proximal support 132 of inner cutting member 130, respectively, such that rotation of outer tube 120 or inner cutting member 130 is not imparted to first or second pushers 557, 559, respectively. However, first and second pushers 557, 559 are translationally coupled with the proximal end portion of outer tube 120 and the proximal end portion of proximal support 132 of inner cutting member 130, respectively, such that translation of first and second pushers 557, 559 similarly translates outer tube 120 and inner cutting member 130, respectively.

With respect to the operation of cam mechanism 550, as protrusion 552 orbits about the longitudinal axis of proximal driver 542 from the initial position illustrated in FIG. 5, protrusion 552 is rotated to contact the second end portion of oblong cam lobe 556 to pivot oblong cam lobe 556 about pivot pin 555 such that the second end portion of oblong cam lobe 556 is urged distally. This distal urging of the second end portion of oblong cam lobe 556, in turn, urges second pusher 559 distally such that inner cutting member 130 is translated distally. Upon further rotation of protrusion 552, protrusion 552 is rotated to contact the first end portion of oblong cam lobe 556 to pivot oblong cam lobe 556 about pivot pin 555 such that the first end portion of oblong cam lobe 556 is urged distally. This distal urging of the first end portion of oblong cam lobe 556, in turn, urges first pusher 557 distally such that outer shaft 120 is translated distally. As inner cutting member 130 and outer tube 120 are sequentially translated distally, housing 548, clevis 554, pivot pin 555, and oblong cam lobe 556, are themselves translated distally via the distal advancement of proximal driver 542. Thus, the above sequential translation of inner cutting member 130 and outer tube 120 is repeated such that inner cutting member 130 and outer tube 120 are alternatingly translated distally during activation.

In addition to the above-detailed alternating distal translation of inner cutting member 130 and outer tube 120, the rotation of proximal driver 542, as detailed above, drives rotation of both inner cutting member 130 and outer tube 120. The rotation and translation of inner cutting member 130 facilitates the boring and engagement of inner cutting member 130 within tissue, while the rotation and translation of outer tube 120 cuts tissue surrounding inner cutting member 130 and captures a sample of tissue within lumen 122 of outer tube 120.

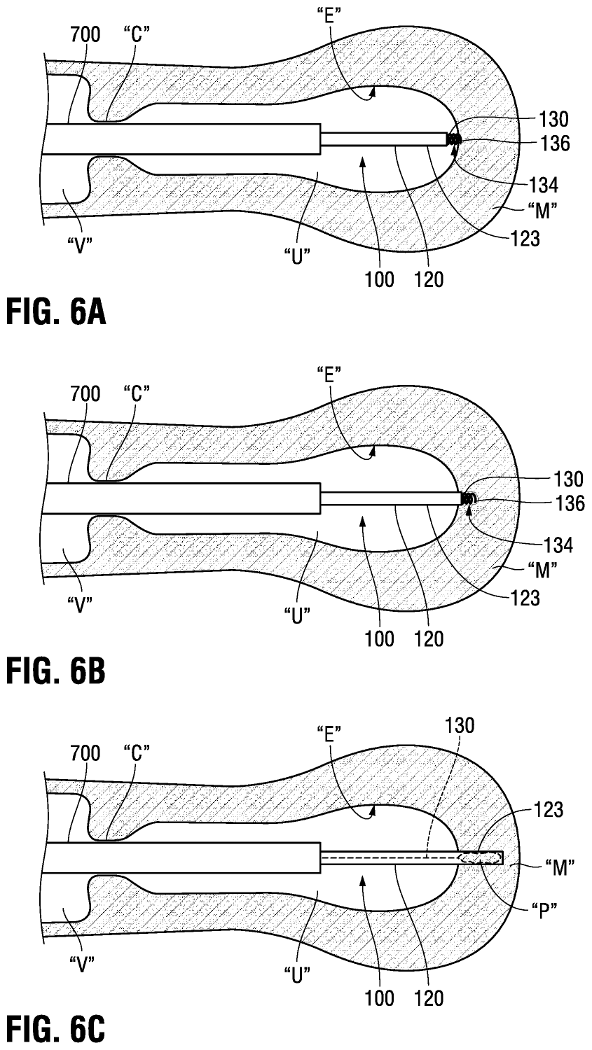

Turning to FIGS. 6A-6C, use of end effector assembly 100 of the present disclosure (whether used with device 10 (FIG. 1), device 20 (FIG. 2), or any other suitable device; whether incorporating inner drive assembly 340 (FIG. 3), inner drive assembly 440 (FIGS. 4A-4C), or inner drive assembly 540 (FIG. 5)) to obtain a biopsy sample is described. Initially, with reference to FIG. 6A, a hysteroscope 700 or other suitable access device may be inserted transvaginally through the vagina “V,” the cervix “C,” and into the uterus “U.” End effector assembly 100, led by the distal end thereof, may then be inserted through a working channel of hysteroscope 700 and into the uterus “U” and manipulated into position such that free distal end 136 of spiral-shaped distal portion 134 of inner cutting member 130 and/or distal end 123 of outer tube 120 are positioned adjacent an area of interest, e.g., adjacent or in contact with endometrial tissue “E.”

Referring to FIGS. 6A and 6B, once the above-noted position has been achieved, end effector assembly 100 is activated e.g., to drive the inner drive assembly thereof (for example, inner drive assembly 340 (FIG. 3), inner drive assembly 440 (FIGS. 4A-4C), or inner drive assembly 540 (FIG. 5)), such that both inner cutting member 130 is driven to rotate and such that inner cutting member 130 is alternatingly translated distally (see, e.g., inner drive assemblies 340 (FIG. 3), 540 (FIG. 5)) or such that inner cutting member 130 is continuously translated distally (see, e.g., inner drive assembly 440 (FIGS. 4A-4C)). These motions enable spiral-shaped distal portion 134 of inner cutting member 130, led by free distal end 136 thereof, to be rotationally and translationally driven through the endometrial tissue “E” and into the myometrial tissue “M.” The spiral-shaped distal portion 134 functions as an anchor to bore into, grasp, and retain the myometrial tissue “M.”

With additional reference to FIG. 6C, in addition to the above-detailed motion of inner cutting member 130, outer tube 120 is also driven to rotate and is translated alternatingly with inner cutting member 130 (see, e.g., inner drive assemblies 340 (FIG. 3), 540 (FIG. 5)) or is periodically translated distally in a step-like manner while inner cutting member 130 is continuously translated (see, e.g., inner drive assembly 440 (FIGS. 4A-4C)). These motions enable outer tube 120, lead by distal edge 124, to be rotationally driven and translated through the endometrial tissue “E” and into the myometrial tissue “M,” cutting out a cylindrical plug of tissue “P,” e.g., about inner cutting member 130, to retain the plug of tissue “P” within lumen 122 of outer tube 120. The plug of tissue “P” serves as the biopsy sample.

Once a sufficient bite of tissue is obtained, end effector assembly 100 may be withdrawn from the surgical site and the obtained biopsy sample, the plug of tissue “P,” may be removed from end effector assembly 100 for analysis.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A tissue biopsy device, comprising:
an inner drive assembly, including:
a drive member configured to receive a rotational input and to rotate in response thereto, the drive member defining a groove along at least a portion of a length thereof, the groove having a continuous configuration and including alternating helical segments and annular segments; and
first and second drive couplers each at least partially engaged within the groove such that the first and second drive coupler are moved through the groove upon rotation of the drive member, the first and second drive couplers being spaced-apart from one another such that when one of the first or second drive couplers is disposed within one of the helical segments of the groove, the other of the first or second drive couplers is disposed within one of the annular segments of the groove, the first and second drive couplers driven to translate longitudinally when moving through one of the helical segments and retained in longitudinal position when moving through one of the annular segments such that the first and second drive couplers are alternatingly translated longitudinally;
an outer tube coupled to the second drive coupler such that translation of the second drive coupler translates the outer tube; and
an inner cutting member extending through the outer tube, the inner cutting member coupled to the first drive coupler such that translation of the first drive coupler translates the inner cutting member.

2. The tissue biopsy device according to claim 1, wherein at least one of the outer tube or the inner cutting member is coupled to the drive member in fixed rotational orientation such that rotation of the drive member drives rotation of the at least one of the outer tube or the inner cutting member.

3. The tissue biopsy device according to claim 1, wherein the tissue cutting member includes a proximal support and a spiral-shaped distal portion extending distally from the proximal support.

4. The tissue biopsy device according to claim 1, further comprising a handle housing supporting a drive assembly therein, the drive assembly configured to connect to the inner drive assembly and to provide the rotational input thereto.

5. The tissue biopsy device according to claim 4, wherein the drive assembly includes a motor configured to provide the rotational input.

6. The tissue biopsy device according to claim 4, further comprising a manual actuator coupled to the drive assembly such that, in response to actuation of the manual actuator, the drive assembly provides the rotational input.

7. A tissue biopsy device, comprising:
a drive extension defining a distal collar;
a stepped clutch, including:
a housing defining an internal cavity and having a plurality of longitudinally-spaced slots defined on an internal wall of the housing surrounding the internal cavity, wherein the drive extension extends into the internal cavity such that the distal collar is disposed within the internal cavity;
a plate disposed within the housing distally of the distal collar and engaged with a first slot of the plurality of longitudinally-spaced slots to retain the plate in position; and
a biasing member disposed between the distal collar and the plate;
an outer tube coupled to the plate such that translation of the plate translates the outer tube; and
an inner cutting member extending through the outer tube, the inner cutting member coupled to the drive extension such that translation of the drive extension translates the inner cutting member,
wherein translation of the drive extension towards the plate loads the biasing member until a sufficient force is achieved that overcomes a retention force of the plate within the first slot such that the biasing member urges the plate to disengage from the first slot, translate distally, and engage with a second slot of the plurality of longitudinally-spaced slots.

8. The tissue biopsy device according to claim 7, further comprising a drive member configured to receive a rotational input and to rotate in response thereto, wherein the outer tube and the inner cutting member are both coupled to the drive member such that rotation of the drive member drives rotation of the outer tube and the inner cutting member.

9. The tissue biopsy device according to claim 8, wherein the drive member is further configured to translate in response to receiving the rotational input, the drive extension coupled to the drive member such that translation of the drive member drives translation of the drive extension.

10. The tissue biopsy device according to claim 9, wherein the drive member includes a helical groove along at least a portion of a length thereof, and wherein a drive coupler is at least partially engaged within the helical groove such that the drive member is translated longitudinally in response to rotation of the drive member.

11. The tissue biopsy device according to claim 8, further comprising a handle housing supporting a drive assembly therein, the drive assembly configured to provide the rotational input to the drive member.

12. The tissue biopsy device according to claim 11, wherein the drive assembly includes a motor configured to provide the rotational input.

13. The tissue biopsy device according to claim 11, further comprising a manual actuator coupled to the drive assembly such that, in response to actuation of the manual actuator, the drive assembly provides the rotational input.

14. The tissue biopsy device according to claim 7, wherein the tissue cutting member includes a proximal support and a spiral-shaped distal portion extending distally from the proximal support.

15. A tissue biopsy device, comprising:
an inner drive assembly, including:
a drive member configured to receive a rotational input and to rotate in response thereto, the drive member defining a helical groove along at least a portion of a length thereof, the drive member including a protrusion extending distally therefrom, the protrusion off-center from a longitudinal axis of the drive member such that the protrusion orbits about the longitudinal axis upon rotation of the drive member about the longitudinal axis;
a drive coupler at least partially engaged within the helical groove such that the drive coupler is moved through the groove upon rotation of the drive member to thereby translate the driver member longitudinally;
an oblong cam lobe positioned distally adjacent the drive coupler, the oblong cam lobe pivotable about a pivot pin transversely aligned on the longitudinal axis; and
first and second pushers positioned distally adjacent the oblong cam lobe, wherein upon orbiting of the protrusion to a first position, the protrusion urges the oblong cam lobe to pivot in a first direction such that a first end portion of the oblong cam lobe urges the first pusher to translate distally and wherein, upon orbiting of the protrusion from the first position to a second position, the protrusion urges the oblong cam lobe to pivot in a second, opposite direction such that a second end portion of the oblong cam lobe urges the second pusher to translate distally;
an outer tube coupled to the second pusher such that translation of the second pusher translates the outer tube; and
an inner cutting member extending through the outer tube, the inner cutting member coupled to the first pusher such that translation of the first pusher translates the inner cutting member.

16. The tissue biopsy device according to claim 15, wherein at least one of the outer tube or the inner cutting member is coupled to the drive member in fixed rotational orientation such that rotation of the drive member drives rotation of the at least one of the outer tube or the inner cutting member.

17. The tissue biopsy device according to claim 15, wherein the tissue cutting member includes a proximal support and a spiral-shaped distal portion extending distally from the proximal support.

18. The tissue biopsy device according to claim 15, further comprising a handle housing supporting a drive assembly therein, the drive assembly configured to connect to the inner drive assembly and to provide the rotational input thereto.

19. The tissue biopsy device according to claim 18, wherein the drive assembly includes a motor configured to provide the rotational input.

20. The tissue biopsy device according to claim 18, further comprising a manual actuator coupled to the drive assembly such that, in response to actuation of the manual actuator, the drive assembly provides the rotational input.
